(19) United States
(12) Patent Application Publication
(10) Pub. No.: US 2013/0202650 A1
(43) Pub. Date: Aug. 8, 2013

(54) TOPICAL COMPOSITION COMPRISING A COMBINATION OF AT LEAST TWO PENETRATION ENHANCING AGENTS

(71) Applicant: Moberg Derma AB, Bromma (SE)

(72) Inventors: Helena Van Den Bussche, Malmo (SE); Christian Palsson, Lund (SE); Johan Borgstrom, Lund (SE); Birgitta Svensson, Limhamn (SE); Anna Holmberg, Lund (SE); Ake Lindahl, Malmo (SE); Bernt Thelin, Lund (SE)

(73) Assignee: Moberg Derma AB, Bromma (SE)

(21) Appl. No.: 13/803,328

(22) Filed: Mar. 14, 2013

Related U.S. Application Data
(63) Continuation of application No. 13/126,261, filed on May 3, 2011.
(60) Provisional application No. 61/109,976, filed on Oct. 31, 2008.

(51) Int. Cl. A61K 47/10; A61K 47/14

(52) U.S. Cl. CPC A61K 47/10; A61K 47/14
USPC 424/278.1

(57) ABSTRACT

The present invention relates to a composition for improved transdermal drug delivery comprising a drug, a combination of at least two penetration enhancing agents, wherein at least on the of the penetration enhancing agents is selected from the group consisting of esters of saturated or unsaturated fatty acids and lower alcohols, and iso-form alcohols; wherein at least one of the penetration enhancing agents is selected from the group consisting of aliphatic diols and triols; and wherein the components are present in a non-aqueous solvent system. A preferred topical composition comprises the active substance imiquimod and the penetration enhancing agents isoproply myristate and propylene glycol.

### CROSS-REFERENCE TO RELATED APPLICATIONS

This application is a continuation of U.S. application Ser. No. 13/126,261, filed May 3, 2011, which is the national stage of PCT/SE2009/051230, filed Oct. 29, 2009, which claims priority to U.S. Provisional Application Ser. No. 61/109,976, filed Oct. 31, 2008, the disclosures of which are each incorporated by reference herein in their entireties.

### TECHNICAL FIELD

The inventions relate to novel compositions for increasing the bioavailability of drugs, and in particular for the delivery of drugs through the skin. The compositions according to the inventions comprise a mixture of at least two penetration enhancers and a non-aqueous solvent system. The inventions also relate to both liquid and solid formulations, such as a gel, a solution, an oil, a lotion, an ointment, a cream or a stick containing the novel composition.

Suitable drugs are drugs exhibiting poor or irregular bioavailability, and examples are found within the group of lipophilic pharmaceutical compounds.

### BACKGROUND

Local application of a drug is preferred in the treatment of diseases of the skin. The reason is, at least theoretically, that the systemic exposure is thereby minimised. This is valid as long as the drug is available to the skin, which requires that the drug is able to penetrate the stratum corneum, the barrier. In general, the amount of drug that is available to the skin, inside the barrier, is in average only a fraction, about 1 to 3%, of the dose applied. If all patients were alike and no scars existed it would not be a problem that 97 to 99% of the dose rests unused outside the barrier. This is however not the case. The permeability of the intact skin differs at least 10 fold between individual patients. Further, scars and other irregularities of the skin are frequent particularly in skin subject to disease, and add to variability in penetration between patients and between different areas of the skin. A 10 fold difference in penetration of a drug may lead to a 10 fold difference in dose, and thus variability in both effect and side effects. For drugs with narrow therapeutic windows, or drugs having side effects, this lack of predictability will result in that some patients receive less than an effective dose, while other patients experience side effects.

If a patient has a scar or in other ways has lost the natural barrier function of the skin, the bioavailability will increase. If the bioavailability with intact barrier function is 1 to 3%, a 30 to 100 fold increase in availability can be expected. Such high increase in the available amount of drug will lead to overdosing by a factor of 30 to 100.

There are several examples of drug products that behave in this way. The solution to the problem is to increase the bioavailability to such extent that a major part of the drug is available. As an example, if the bioavailability of a drug is 50% with intact barrier function, the maximum systemic exposure will be 2 times the intended dose when the barrier function is deficient.

U.S. Pat. No. 6,121,314 A (NOVARTIS AG); U.S. Pat. No. 6,005,001 A (NOVARTIS AG); and U.S. Pat. No. 5,856,355 A (NOVARTIS AG) disclose “non-greasy topical solutions, emulsion gels or lotions comprising as the active agent a compound of formula I and a lower alkanol, and if desired together with a solubilizing agent or an oil phase such as isopropyl myristate are useful delivery systems” for the delivery of terbinafine. These documents do not disclose or suggest the need for the combination of two enhancers to achieve improved penetration enhancing properties.

GB 2146528 A (HOWELLS TREVOR) discloses compositions for treatment of the skin or the scalp to promote hair growth comprising a moisturiser e.g. isopropyl myristate, an oil e.g. lanolin, an emulsifier e.g. Sorbitol ester, a preservative e.g. Nipastat, a follicle stimulant e.g. Tabasheer, and an enzyme catalyst e.g. carboxylase dehydrase obtained from the excretion of a gastropod, especially Helix aspersa. This document does not contain any information on the positive effects on penetration of isopropyl myristate on its own or in combination with other agents.

DE 4038385 A (ROECAR HOLDINGS NV) discloses sitosterol and its glycosides having improved bioavailability in the form of microemulsions using lecithin as the carrier. Preferably, these microemulsions contain isopropyl myristate as emulsifier and isopropanol as co-emulsifier. The microemulsions consist of 21-23% lecithin, 15-16% isopropanol and 7.0-7.5% isopropyl myristate in water as the dispersant. When sitosterol and its glycosides are in the form of microemulsions using lecithin as carrier, the bioavailability is improved. This document does not teach enhanced penetration using the combination of two different penetration enhancers in water-free formulations.

U.S. Pat. No. 6,503,894 B (UNIMED PHARMACEUTICALS INC); US 2002183296 A (DUDLEY ROBERT ET AL.); US 2003022877 A (DUDLEY ROBERT); US 2003139384 A (DUDLEY, ROBERT) and US 2003232072 A (DUDLEY, ROBERT ET AL.) as representatives for a large patent family describe a composition comprising an androgenic or anabolic steroid, a C₁-C₄ alcohol, a penetration enhancer such as isopropyl myristate, and water, together with a gelling agent forming a hydroalcoholic gel formulation. These documents disclose enhanced penetration caused by isopropyl myristate but not in combination with other agents. All formulations in these documents contain water.

WO 2005/025626 A (PROCTER) discloses microcapsules for controlled release. In claim 4, the stabilizer is selected from the group consisting of isopropyl myristate and several other agents. This document teaches the use of isopropyl myristate as a stabiliser for microcapsules.

WO 97/34644 A (HOECHST AG) discloses formulations suitable for the treatment of nail psoriasis and containing a substance effective against psoriasis, at least one spreading solvent including isopropyl myristate, at least one readily volatile solvent and a film-forming agent. There is no teaching of the penetration enhancing properties of isopropyl myristate alone or in combination with other agents in this reference.

EP 1889609 A (MEDA AB) mentions the use of isopropyl myristate as an emollient in aqueous foam formulations containing fatty acids. There is no teaching of the penetration enhancing effect of the combination of isopropyl myristate alone or in combination with other excipients.

U.S. Pat. No. 7,425,340 A (ANTARES PHARMA IPL AG) discloses the use of alcohols as penetration enhancers for a combination of a urea compound with an anticholinergic or antispasmodic agent. This document does not teach the usefulness and the results of a combination of enhancers.

The influence of isopropyl myristate (IPM), isopropyl alcohol (IPA) and a combination of both was studied in view of hydrocortisone (HC) permeation across the human stratum corneum (SC) by Brinkmann et al (BRINKMANN, I, et al. Role of isopropyl myristate, isopropyl alcohol and a combination of both in hydrocortisone permeation across the human stratum corneum. Skin Pharmacol Appl Skin Physiol. 2003, vol. 16, no. 6, p. 393-404). IPM and IPA and their combination were incorporated into water-containing hydrophilic ointment (WHS), and the resulting effects on HC permeation and on HC accumulation in human SC were investigated as well as the influence of these substances on the microstructure of the SC. Differential scanning calorimetry as well as wide- and small-angle X-ray diffraction show that IPM incorporation into SC results in densely packed bilayer lipids and a loss of order of the corneocyte-bonded lipids. Both effects result in a decreased diffusion coefficient of HC in SC and thus in a decreased permeation rate compared to that of HC from WHS.

In another article, Brinkmann et al (BRINKMANN, I, et al. An attempt to clarify the influence of glycerol, propylene glycol, isopropyl myristate and a combination of propylene glycol and isopropyl myristate on human stratum corneum. Pharmazie. 2005, vol. 60, no. 3, p. 215-220) also investigated the effect on stratum corneum of combinations of propylene glycol and IPM. These investigations resulted in a conclusion where IPM alone decreased penetration of drugs from a WHS formulation while combinations with isopropyl alcohol increased penetration.

Gorukanti et al (GORUKANTI, S. R., et al. Transdermal delivery of antiparkinsonian agent, benztropine. I. Effect of vehicles on skin permeation. Int J Pharm. 1999, vol. 192, no. 2, p. 159-172) have demonstrated increased penetration of benztropine (BZ) free base and its mesylate salt in mouse when using a formulation containing IPM and alcohols (ethanol, IPA and tertiary butyl alcohol). A tBtOH-IPM (2:8) combination produced the highest BZ flux from the mesylate salt, i.e., 2016 mg per cm(2) h(−1), which was 100-fold greater than from water and 44-540-fold greater than the individual neat solvents, respectively. The observed permeation enhancement of BZ mesylate by the alkanol-IPM mixtures was probably as a result of a combination of decreasing barrier ability of the stratum corneum by the binary vehicles and moderately partitioning BZ mesylate through the viable epidermis/dermis.

Panchagnula et al (PANCHAGNULA, R., et al. Feasibility studies of dermal delivery of paclitaxel with binary combinations of ethanol and isopropyl myristate: role of solubility, partitioning and lipid bilayer perturbation. Farmaco. 2005, vol. 60, no. 11-12, p. 894-9) demonstrated the positive effect on penetration of IPM in combination with ethanol.

Cha et al (CHA, B. J., et al. Enhanced skin permeation of a new capsaicin derivative (DA-5018) from a binary vehicle system composed of isopropyl myristate and ethoxydiglycol. Arch Pharm Res. 2001, vol. 24, no. 3, p. 224-8) demonstrated a negative effect of oleic acid on the IPM mediated penetration of DA-5018 through skin.

Aranello et al investigated the effect of IPM and PG on the penetration of diclofenac through skin (ARANELLO, A, et al. Influence of propylene glycol and isopropyl myristate on the in vitro percutaneous penetration of diclofenac sodium from carbopol gels. Eur J Pharm Sci. 1999 January, vol. 7, no. 2, p. 129-35.) None of the combinations disclosed below were studied in a water-free environment in this paper.

In another paper (LARRUCEA, E, et al. Combined effect of oleic acid and propylene glycol on the percutaneous penetration of tenoxicam and its retention in the skin. Eur J Pham Biopharm. 2001 September, vol. 52, no. 2, p. 113-9) the combined effect of oleic acid and propylene glycol on percutaneus penetration of tenoxicam was studied. There is no teaching regarding the use of water-free formulations.

US 2007269393 A (WEPFER SCOTT) discloses a topical anesthetic formulation in the form of an anhydrous gel comprising benzyl alcohol, propylene glycol, and ethoxydiglycol as skin penetration enhancing agents.

US 2007179121 A (PLOTT R T) concerns a composition comprising a corticosteroid; two or more penetration enhancers selected from the group consisting of diisopropyl adipate, dimethyl isosorbide, propylene glycol, 1,2,6-hexapetriol, and benzyl alcohol.

US 2008153885 A (MEADOWS CHEYNEY ET AL.) concerns a transdermal liquid preparation comprising a first and a second dermal penetration enhancer, an aprotic primary solvent, and a therapeutically effective amount of flunixin or a pharmaceutically acceptable salt thereof.

US 2008260655 A (TAMARKIN DOV ET AL.) relates to stable substantially non-aqueous, non-alcoholic, non-silicone, foamable carrier compositions comprising petrolatum or mixtures thereof, at least one foam agent, at least one propellant, and with and without the addition of an active agent.

U.S. Pat. No. 5,837,289 A (GRASELA JOHN ET AL.) describes the use of two separate penetration enhancers in a topical formulation, where the first penetration enhancer preferably is a lecithin organogel formed with isopropyl palmitate or isopropyl myristate, and the second penetration enhancer preferably is a polyoxymer, preferably a polyoxyalkylene derivative of propylene glycol.

WO 2007/103555 A (NUVIANCE INC) concerns topical compositions for the treatment of skin ailments, comprising two or more transdermal penetrants working synergistically but by disparate biochemical pathways.

WO 2007/019224 A (WATSON LABORATORIES INC) relates to methods and formulations of enhancing the permeability of the skin of a subject to a drug, including administering a combination of lauryl alcohol and isopropyl myristate as a penetration enhancer to the area of skin to provide synergistically enhanced penetration of the drug.

WO 2007/066149 A (PHARMAKODEX LTD) relates to compositions and applicator devices for providing accurate and localized administration of pharmaceutical compositions containing therapeutic agents to the skin.

### SUMMARY OF INVENTION

The problems described above are now solved for a number of drug substances by a surprising effect that is generated when the following conditions are at hand.

A general embodiment of the inventions is a topical composition comprising a drug, a combination of at least two penetration enhancing agents, wherein at least one of the penetration enhancing agents is selected from the group consisting of esters of saturated or unsaturated fatty acids and lower alcohols, and iso-forms of alcohols; wherein at least one of the penetration enhancing agents is selected from the group consisting of aliphatic diols and triols; and wherein the components are present in a non-aqueous solvent system.

In this general embodiment, the drug is preferably a lipophilic drug, more preferably a lipophilic drug chosen from the group consisting of immunomodulators or immune response modifiers, tricyclic antidepressants, analgetics, anaestetics, anti-inflammatory, β blocking agents, antimicrobials and Ca blocking agents, most preferably an immunomodulating compound, such as toll like receptor 7(TLR7) ligand, for example imiquimod.

Although the components used in the present compositions are standard components that have been used in topical products, novel combinations of these components demonstrate a surprisingly high effect on the delivery of the drug substance over skin in “in vitro” studies.

### BRIEF DESCRIPTION OF DRAWINGS

FIG. 1 illustrates the results of Example 1;

FIG. 2 illustrates the results of Example 2;

FIG. 3 illustrates the results of Example 3;

FIG. 4 illustrates the results of Example 5;

FIG. 5 illustrates the results of Example 6; and

FIG. 6 illustrates the results of Example 7.

### DESCRIPTION OF EMBODIMENTS

The following description is of the best mode presently contemplated for carrying out the inventions. This description is not to be taken in a limiting sense, but is made solely for the purpose of describing the general principles of the inventions. The scope of the inventions should be determined with reference to the claims.

Before the inventions are described in detail, it is to be understood that these inventions are not limited to the particular compounds described or process steps of the methods described as such compounds and methods may vary. It is also to be understood that the terminology used herein is for purposes of describing particular embodiments only, and is not intended to be limiting. It must be noted that, as used in the specification and the appended claims, the singular forms “a,” “an” and “the” include plural referents unless the context clearly dictates otherwise.

Further, the term “about” is used to indicate a deviation of +/−10% of the given value, preferably +/−5% and most preferably +/−2% of the numeric values, when applicable.

The term “non-aqueous” solvent system means that no water is added in the present invention. The terms “water-free” and “non-aqueous” do not exclude the presence of trace amounts of water present in the starting materials, but make it clear that no water is added to the composition.

A substance considered to be lipophilic when it has an affinity for fat and high lipid solubility. Lipophilicity is thus a physicochemical property which describes the partitioning equilibrium of solute molecules between water and an immiscible organic solvent, favoring the latter.

Lipophilicity is generally expressed by the partition, Log P, between water and a water-immiscible solvent. The solvent most commonly used in drug discovery and development is 1-octanol. Log P refers to the logarithm of the Partition Coefficient, P, which is defined as the ratio of concentration of neutral species in octanol divided the concentration of neutral species in water. A lipophilic drug suitable for the purposes of this application is a drug having a log P of at least about 1.5.

The present inventors make available a topical composition comprising a drug, a combination of at least two penetration enhancing agents, wherein at least one of the penetration enhancing agents is selected from the group consisting of esters of saturated or unsaturated fatty acids and lower alcohols, and iso-forms of alcohols; wherein at least one of the penetration enhancing agents is selected from the group consisting of aliphatic diols and triols; and wherein the components are present in a non-aqueous solvent system.

The present formulations thus contain a combination of at least two penetration enhancers and at least one non-aqueous solvent. The formulations may optionally contain at least one solubility modifier and it is also understood that the formulation will contain excipients normally used in formulations intended for topical use in order to create a suitable product.

The penetration enhancer combinations suitable for the performance of the formulations, measured as delivery of drug over a skin membrane, are preferably a mixture at least one penetration enhancer chosen from the group of esters of saturated or unsaturated fatty acids and lower alcohols or iso-forms of alcohols, such as isopropyl alcohol, isobutyl alcohol and, and at least one penetration enhancer chosen from the group of aliphatic diols and triols. Non-limiting examples of penetration enhancers of the ester type are methyl laurate, isopropyl myristate, isopropyl palmitate, butyl stearate, ethyl oleate, and di-isopropyl adipate. Sorbic esters of fatty acids and monoglycerides, as well as mono-, di- and tri-unsaturated fatty acid esters, have also been used. Further non-limiting examples of the penetration enhancers of the aliphatic diols and triols type include ethylene glycol, propylene glycol, butylene glycol, hexylene glycol and glycerol.

The drug substances suitable for the novel formulations are preferably lipophilic or soluble in a non-aqueous environment, and preferably a drug having a log P in the interval of about 1.5 to about 5.

Furthermore, the most preferred drug substances are potent and/or toxic lipophilic compounds with a small or narrow therapeutic window. Thus, said drug is preferably a lipophilic drug, and more preferably a lipophilic drug chosen from the group consisting of immunomodulators or immune response modifiers, tricyclic antidepressants, analgetics, anaestetics, anti-inflammatory, β blocking agents, antimicrobials and Ca blocking agents.

More preferably said drug comprises an immunomodulating compound, and according to one embodiment, said immunomodulating compound comprises a toll like receptor 7(TLR7) ligand. Examples include, but are not limited to: imiquimod; amlodipine; nifedipine; felodipine; and resiquimod.

A non-limiting example of an immunomodulating compound is imiquimod.

The drug or drugs will be present in an amount needed to generate a pharmacological effect in the targeted tissue, the skin. According to an embodiment of the inventions, said drug is present in an amount of about 0.01 to about 5% by weight based on the total weight of the composition. Preferably, as a result of the improved penetration obtained by the inventive formulation, said drug is present in a lower amount, for example in an amount of about 0.01 to about 1% by weight based on the total weight of the composition.

According to an embodiment of the invention, freely combinable with the other embodiments, the alcohol is isopropyl alcohol or isobutyl alcohol.

According to a further embodiment of the invention, the penetration enhancing agents comprise isopropyl myristate and propylene glycol.

According to an embodiment, the combined amount of penetration enhancers is from about 1 to about 99% by weight based on the total weight of the composition. In a liquid formulation, such as a gel, a cream, an ointment, or an oily formulation or oil, the combined amount of penetration enhancers is preferably from about 50 to about 99% by weight based on the total weight of the composition. In a solid or semisolid formulation, such as a paste or a stick, the combined amount of penetration enhancers is preferably from about 5 to 60% by weight based on the total weight of the composition.

In an embodiment of the inventions, a ratio between first and second penetration enhancing agents is from about 1:10 to about 10:1, and preferably about 1:2 to about 2:1.

The solvent, or mixture of solvents, suitable in this formulation system are taken from the group of compounds exemplified by aromatic alcohols such as benzyl alcohol, esters of alpha-hydroxy acids such as but not limited to esters of short-chain alcohols, having up to eight carbons, and lactic acids or fatty acids. Other types of suitable solvents, having similar solubility properties, are represented by cyclohexanol, diacetone alcohol, ethylene glycol monomethyl ether, diethylene glycol monomethyl ether, ethylene glycol monoethyl ether, diethylene glycol monoethyl ether, methyl salicylate, benzoic acid, oleyl alcohol, and acetic acid. Short chain aliphatic alcohols such as ethanol, propanol and isopropanol are also suitable. Preferably, the solvent is present in an amount of 1 to 80 weight %, based on the total weight of the composition.

According to a preferred embodiment of the inventions, the non-aqueous solvent comprises at least one selected from the group consisting of aromatic alcohols, esters of aromatic alcohols and fatty acids or esters of alpha-hydroxy acids, and short-chain alcohols having up to eight carbons. According to a non-limiting example, currently preferred by the inventors, the alcohol is benzyl alcohol.

Preferably the non-aqueous solvent comprises at least one selected from the group consisting of cyclohexanol, diacetone alcohol, ethylene glycol monomethyl ether, diethylene glycol monomethyl ether, ethylene glycol monoethyl ether, diethylene glycol monoethyl ether, methyl salicylate, benzoic acid, oleyl alcohol, and acetic acid. According to a non-limiting example, currently preferred by the inventors, the esters are chosen among lactic acid esters, and the currently preferred solvents are chosen from methyl lactate, ethyl lactate, propyl lactate and butyl lactate.

According to another embodiment, the solvent comprises at least one selected from the group consisting of short chain aliphatic alcohols having up to eight carbons. Preferably said solvent comprises at least one selected from the group consisting of ethanol, propanol and isopropanol.

According to another embodiment, said solvent comprises at least one solvent selected from the group comprising esters of alpha-hydroxy acids and short-chain alcohols having up to eight carbons.

According to any one of the above embodiments, said solvent or combinations of solvents are present in an amount of 1 to 80% by weight based on the total weight of the formulation.

A composition according to an embodiment of the inventions may also comprise a structural agent. The topical composition can for example be diluted with non-active components normally used in topical products in order to create texture and other physical properties. Non-limiting examples of such components are mineral oil, soft white paraffin and waxes, exemplified by but not limited to carnauba wax.

Said structural agent preferably comprises at least one wax selected from the group comprising soft white paraffin, paraffin oil and waxes, exemplified by but not limited to carnauba wax, to increase the viscosity of the formulation. Alternatively or in combination therewith, the structural agent may comprise a soluble or non-soluble polymer. Examples of non-soluble polymers are e.g. polymers of latex type, such as but not limited to Eudragit® (Evonik Industries).

The composition further preferably comprises a colouring agent. A colouring agent is useful in indicating the amount and distribution of the composition, when applied to the skin. Therefore a coloured composition is easier to dose and apply evenly. A skilled person is capable of selecting a suitable colouring agent or pigment among those approved for use in topical pharmaceutical compositions.

The ideal solvent(s) in the formulation can be selected based on solubility properties. A highly effective solvent, in this formulation, such as benzyl alcohol, can be replaced with other(s) under the condition that the solvents used have similar solubility parameters with respect to hydrogen binding, polar and dispersion forces. With similar solubility parameters a variation within ±10% is intended.

Solubility modifiers represented by carboxylic acids, including fatty acids, can be exemplified by, but not limited to, caprylic acid, capric acid, lauric acid, myristic acid, palmitic acid, stearic acid, arachidic acid, behenic acid, lignoceric acid, myristoleic acid, palmitoleic acid, linoleic acid, linolenic acid and isostearic acid, oleic acid, benzoic acid, acetic acid, citric acid, oxalic acid salicylic acid ascorbic acid, and alpha-hydroxy acids such as glycolic acid, lactic acid, malic acid and tartaric acid. The role of solubility modifiers is to adjust the solubility of the drug so that the formulation gets a suitable degree of saturation. According to any one of the above embodiments, the composition may further comprise a solubility modifier in amounts of about 1 to about 60% by weight based on the total weight of the composition.

A non-limiting example of a solubility modifier currently preferred by the inventors is oleic acid.

A topical composition according to the inventions can be formulated as a solution, a gel, a cream, a paste, an ointment, an oil, or a stick. Persons skilled in the art of topical pharmaceutical formulations can easily compose a solution, a gel, a cream, a paste, an ointment, an oil or a stick within the boundaries of the claims without inventive effort.

The composition can be in the form of a stick. Sticks are well known and any known method for manufacturing sticks using the present composition can be used. An example of a method that can be used is disclosed in U.S. Pat. No. 4,069,574 (Krevald), the complete disclosure of which is incorporated by reference. An example a suitable stick is disclosed in U.S. Pat. No. 5,819,993 (Wile), the complete disclosure of which is incorporated by reference.

When the composition is used in stick form, preferably the total amount of penetration enhancers is about 1 to 50 weight %, based on the total weight of the composition.

The improved penetration obtained by the present inventors offers many advantages. It is for example an advantage of the inventions that the penetration of a drug can be improved using penetration enhancers and solvents approved for pharmaceutical use. Further advantages of the inventions will become apparent to persons skilled in the art upon a closer study of the description, claims and non-limiting examples.

### EXAMPLES

The present invention will be better understood in connection with the following examples, which are intended as an illustration of and not a limitation upon the scope of the invention.

The in vitro evaluation of pharmaceutical formulations, as referred to in the examples below, was conducted at 32° C., with approximately 900 μm thick full-thickness pig ear skin membranes, using Bronaugh diffusion cells with a cell area of 0.63 cm². In example 4, a Franz diffusion apparatus was used. The receptor media flow was set to 1.6 ml/h and a citrate buffer solution or a phosphate buffered saline solution containing a surfactant was used as receptor medium.

Imiquimod (3-(2-methylpropyl)-3,5,8-triazatricyclo[7.4.0.0^{2,6}]trideca-1(9),2(6),4,7,10,12-hexaen-7-amine), is an immune response modifier approved to treat actinic keratosis, superficial basal cell carcinoma and external genital warts or condyloma, currently marketed under the trade names Aldara™ and Beselna™. Imiquimod was chosen as side effects have been reported, ranging from local reactions such as redness, skin peeling, flaking, swelling, crusting, itching/burning, to systemic effects, such as fever, muscle aches etc. Imiquimod was also chosen for the purpose of exemplifying the inventions, as there is a commercial formulation (Aldara™) available for comparative experiments.

### Example 1

The in vitro penetration of imiquimod from Formulation A, presented in Table 1, was evaluated and compared to the penetration of imiquimod from the commercially available Aldara™ 5% cream (3M Health Care Limited).

TABLE 1
Composition of Formulation A
Formulation A

| Ingredient | Function | % (w/w) |
|---|---|---|
| Imiquimod | drug | 0.25 |
| Benzyl alcohol | S | 49.75 |
| Propylene glycol | PE | 50.00 |

| | |
|---|---|
| Total % (w/w) | 100 |

The abbreviation “S” denotes solvent and “PE” penetration enhancer.

The experimental parameters of the in vitro experiment are presented in Table 2.

TABLE 2
Experimental parameters
Example 1

| Total experimental time (h) | 48 | |
|---|---|---|
| Sampling points (h) | 12, 24, 36, 48 | |
| Receptor medium | Citrate buffer, pH 3.7 | |
| | Formulation A | Aldara ™ 5% cream |
| Number of cells | 7 | 7 |
| Mean amount formulation | 59.7 | 60.6 |
| applied per cell (mg) | | |

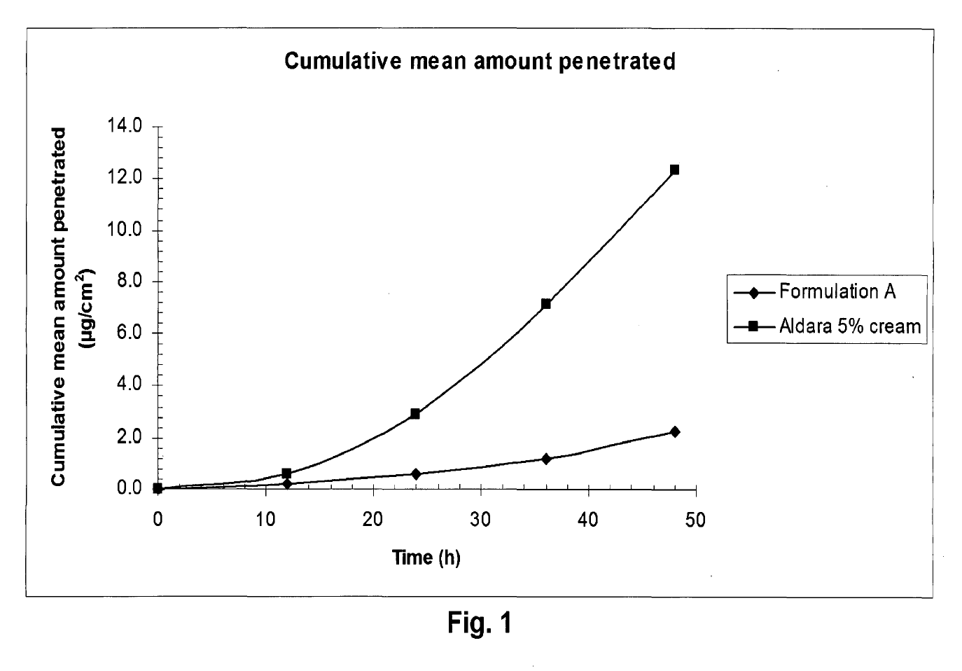

The results presented in FIG. 1 show that the penetrated average amount of imiquimod after 48 hours was larger for Aldara™ than for Formulation A, but the skin penetration is in the same order of magnitude. The mean amount imiquimod penetrated per cm², n=7. Formulation A contains imiquimod, benzyl alcohol and propylene glycol.

### Example 2

The in vitro penetration of imiquimod from Formulation B, presented in Table 3, was evaluated and compared to the penetration of imiquimod from the commercially available Aldara™ 5% cream (3M Health Care Limited).

TABLE 3
Composition of Formulation B
Formulation B

| Ingredient | Function | % (w/w) |
|---|---|---|
| Imiquimod | drug | 1.50 |
| Benzyl alcohol | S | 42.80 |
| Propylene glycol | PE | 18.40 |
| Oleic acid | SM | 7.10 |
| Isopropyl myristate | PE | 30.20 |

| | |
|---|---|
| Total % (w/w) | 100.00 |

The following abbreviations are used: “S” solvent, “PE” penetration enhancer, and “SM” solubility modifier.

The experimental parameters of the in vitro experiment are presented in Table 4.

TABLE 4
Experimental parameters
Example 2

| Total experimental time (h) | 48 | |
|---|---|---|
| Sampling points (h) | 6, 12, 24, 36, 48 | |
| Receptor medium | PBS buffer, pH 7.4 and Volpo | |
| | Formulation B | Aldara ™ 5% cream |
| Number of cells | 5 | 4 |
| Mean amount formulation | 62.9 | 62.6 |
| applied per cell (mg) | | |

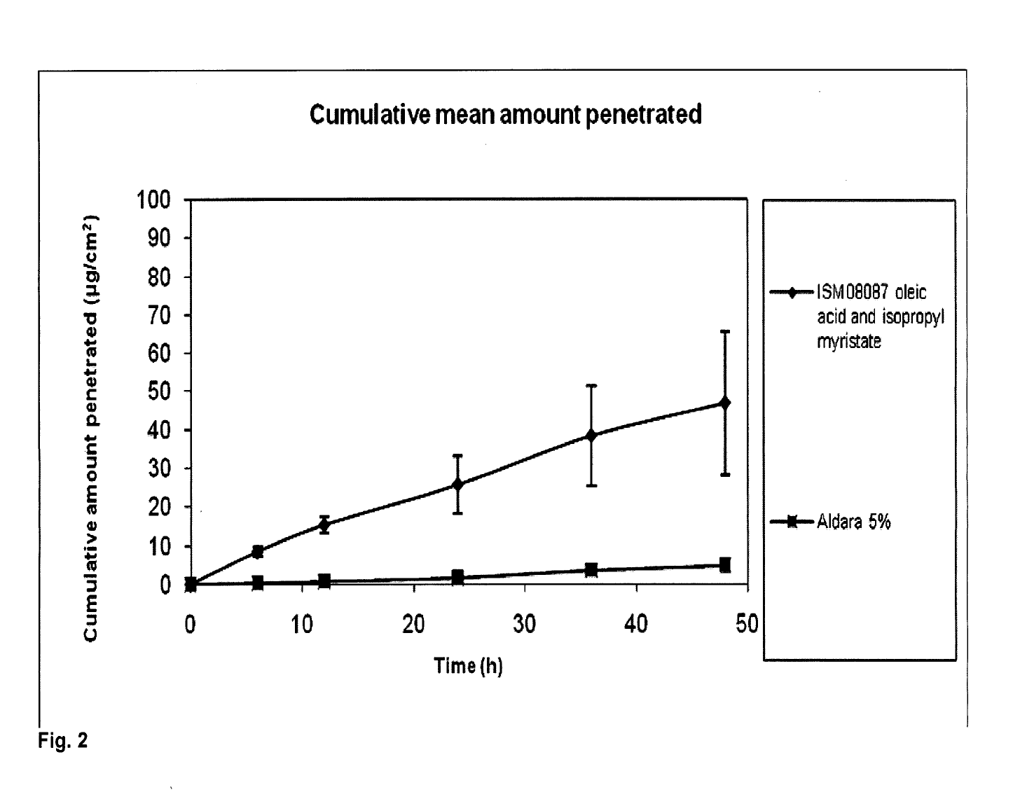

The results, presented in FIG. 2, show that the mean amount imiquimod penetrated was more than 45 times higher using Formulation B compared to the Aldara™ 5% cream. FIG. 2 shows the mean amount imiquimod penetrated per cm², n=7. Formulation B contains imiquimod, benzyl alcohol, propylene glycol, oleic acid and isopropyl myristate.

Comparing these results with the results presented in Example 1, it can be seen that a very high penetration increase was obtained when oleic acid and isopropyl myristate were added to the formulation. For Formulation B, the dose fraction imiquimod penetrated after 48 hours was approximately 5% (w/w) while for Aldara™ 5% cream it was only about 0.1% (w/w).

### Example 3

The in vitro penetration of imiquimod from Formulation C and Formulation D, presented in Table 5, was evaluated. The penetration was then compared to the penetration of imiquimod from the commercially available Aldara™ 5% cream (3M Health Care Limited).

TABLE 5
Compositions of Formulations C and D

| | | Formulation C | Formulation D |
|---|---|---|---|
| Ingredient | Function | % (w/w) | % (w/w) |
| Imiquimod | drug | 0.25 | 0.17 |
| Benzyl alcohol | S | 49.68 | 48.66 |
| Propylene glycol | PE | 50.06 | 20.96 |
| Isopropyl myristate | PE | — | 30.22 |

| | | |
|---|---|---|
| Total % (w/w) | 100.00 | 100.00 |

The experimental parameters of the in vitro experiment are presented in Table 6.

TABLE 6
Experimental parameters
Example 3

| Total experimental | 24 | | |
|---|---|---|---|
| time (h) | | | |
| Sampling points (h) | 6, 12, 24 | | |
| Receptor medium | Phosphate buffered saline, pH 7.4 + | | |
| | 6% PEG-20 Oleyl ether | | |
| | | | Aldara ™ 5% |
| | Formulation C | Formulation D | cream |
| Number of cells | 7 | 6 | 7 |
| Mean amount | 61.1 | 60.1 | 63.3 |
| formulation applied | | | |
| per cell (mg) | | | |

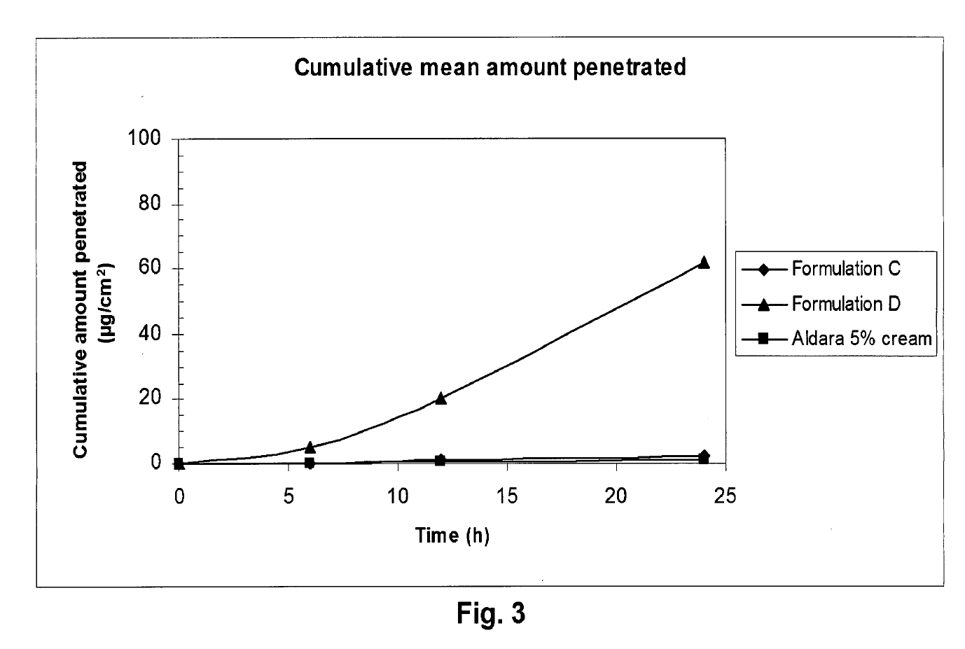

The results, presented in FIG. 3, show that the mean amount imiquimod penetrated from Formulation D was more than 25 times higher than the corresponding penetration from Formulation C.

FIG. 3 shows the mean amount imiquimod penetrated per cm², n=7 for Formulation C, n=6 for Formulation D and n=7 for Aldara™ 5% cream. Formulation C contains imiquimod, benzyl alcohol and propylene glycol, Formulation D contains imiquimod, benzyl alcohol, propylene glycol and isopropyl myristate. These results demonstrate that the presence of isopropyl myristate and benzyl alcohol promotes a high penetration of imiquimod. The dose fraction imiquimod penetrated from Formulation C and Formulation D was 0.7% (w/w) and 37.5% (w/w), respectively, while for Aldara™ 5% cream the dose fraction penetrated was less than 0.1% (w/w).

### Example 4

Two formulations, including benzyl alcohol and isopropyl myristate, but with and without propylene glycol, were tested for drug penetration trough full thickness pig ear skin in a Franz cell diffusion apparatus. The formulations are presented in Table 7.

TABLE 7
Compositions of Formulations D and E

| | | Formulation D | Formulation E |
|---|---|---|---|
| | | ISM08180 | ISM08076 |
| Ingredient | Fuction | % (w/w) | % (w/w) |
| Imiquimod | drug | 0.17 | 0.10 |
| Benzyl alcohol | S | 48.8 | 49.9 |
| Propylene glycol | PE | 21.0 | — |
| Isopropyl myristate | PE | 30.0 | 50.0 |

| | | |
|---|---|---|
| Total % (w/w) | 100.00 | 100.00 |

The average fraction of Imiquimod that penetrated from formulation D was 15.2%, while the penetrated fraction from formulation E was only 1.0%. This clearly shows the importance of the presence of propylene glycol in the formulations according to the innovations.

TABLE 8
Experimental parameters
Example 4

| Total experimental time (h) | 16 | |
|---|---|---|
| Sampling points (h) | 16 | |
| Receptor medium | Phosphate buffered saline, pH 7.4 + | |
| | 6% PEG-20 oleyl ether | |
| | Formulation D | Formulation E |
| Number of cells | 3 | 4 |
| Mean amount formulation | 89.7 | 89.8 |
| applied per cell (mg) | | |

### Example 5

The solvents in the formulation can be selected based on solubility properties. A highly effective solvent, in this formulation, such as benzyl alcohol can be replaced with other(s) under the condition that the solvents used have similar solubility properties. In this example benzyl alcohol has been replaced with the butyl ester of lactic acid. The composition of the formulations F and G are presented in Table 9.

TABLE 9
Compositions of Formulations F and G

| | | Formulation F | Formulation G |
|---|---|---|---|
| Ingredient | Function | % (w/w) | % (w/w) |
| Imiquimod | drug | 0.09 | 0.165 |
| Benzyl alcohol | S | — | 49 |
| Butyl lactate | S | 40 | — |
| Propylene glycol | PE | 29.89 | 28.82 |
| Isopropyl myristate | PE | 30 | 21 |
| Butyl hydroxyanisol | antioxidant | 0.02 | 0.02 |

| | | |
|---|---|---|
| Total % (w/w) | 100.00 | 100.00 |

The experimental conditions are presented in Table 10.

TABLE 10
In vitro experiment
Example 5

| Total experimental | 24 | | |
|---|---|---|---|
| time (h) | | | |
| Sampling points (h) | 24 | | |
| Receptor medium | PBS buffer, pH 7.4 and Volpo 6% (PEG-20 | | |
| | oleyl ether) | | |
| | Formulation F | Formulation G | Aldara ™ 5% |
| Number of cells | 5 | 4 | 4 |
| Mean amount | 51.4 | 51.3 | 52.0 |
| formulation applied | | | |
| per cell (mg) | | | |

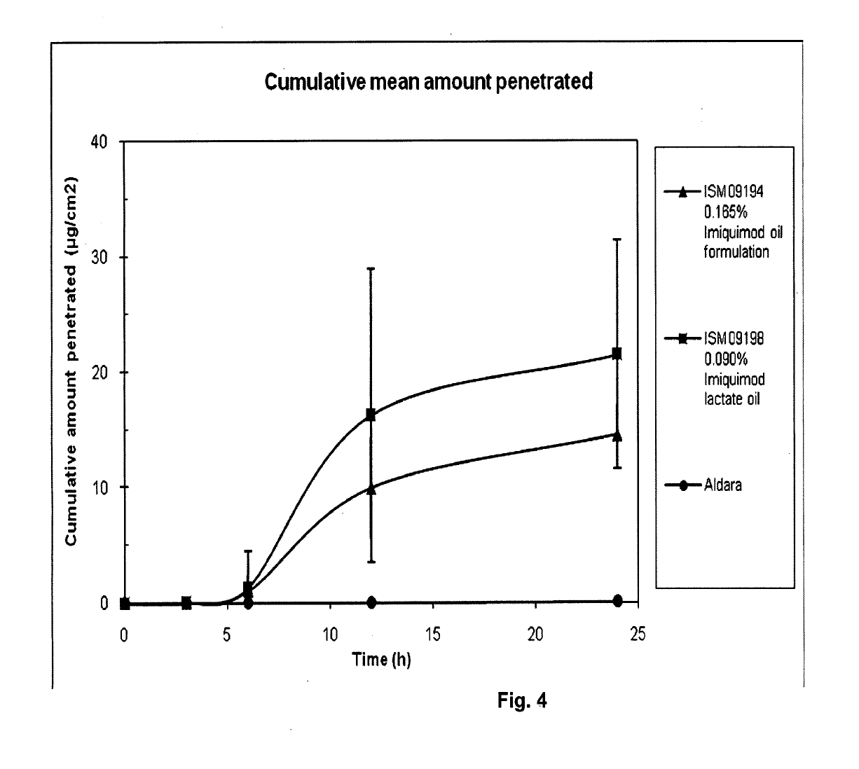

Aldara™ cream was used as comparator in the skin penetration study. The amount of imiquimod present in dermis was determined in this study. After 20 hours of exposure to the products F and G as well as Aldara™, the skin membranes were washed and stratum corneum was removed. The resulting dermal tissue was investigated for presence of imiquimod. While formulations F and G produced dermal concentrations of 62 and 59 μg/ml respectively the commercial product Aldara™ only produced a tissue concentration of 7 μg/ml. The cumulative amount penetrated imiquimod is shown in Table 11 and FIG. 4.

The penetration of imiquimod into skin is similar for formulations F and G demonstrating the possibility of exchanging benzyl alcohol with other solvents based on solubility property criteria.

TABLE 11
Cumulative amount penetrated imiquimod (ISM09199)

| Fraction | | | | |
|---|---|---|---|---|
| F0 | F1 | F2 | F3 | F4 |
| Sampling time fraction (h) | | | | |
| 0 | 6 | 8 | 10 | 12 |

| | | |
|---|---|---|
| Sample | Cell | Cumulative amount penetrated (μg/cm²) |

| | | | | | | |
|---|---|---|---|---|---|---|
| ISM09194 | 1 | 0.000 | 1.637 | 3.723 | 5.612 | 7.506 |
| | 2 | 0.000 | 0.000 | 0.235 | 1.200 | 3.536 |
| | 3 | 0.000 | 3.316 | 6.741 | 10.864 | 16.624 |
| | 4 | 0.000 | 0.353 | 0.960 | 3.197 | 5.140 |
| Mean | | 0.00 | 1.33 | 2.91 | 5.22 | 8.20 |
| SD | | 0.00 | 1.50 | 2.96 | 4.17 | 5.85 |
| ISM09198 | 5 | 0.000 | 0.259 | 0.481 | 1.039 | 3.142 |
| | 6 | 0.000 | 0.000 | 0.115 | 0.296 | 0.612 |
| | 7 | 0.000 | 0.000 | 0.253 | 0.915 | 3.355 |
| | 8 | 0.000 | 0.000 | 0.063 | 0.180 | 0.285 |
| | 9 | 0.000 | 0.215 | 0.582 | 1.413 | 4.074 |
| Mean | | 0.00 | 0.09 | 0.30 | 0.77 | 2.29 |
| SD | | 0.00 | 0.13 | 0.23 | 0.52 | 1.72 |
| Aldara ™ | 10 | 0.000 | 0.000 | 0.000 | 0.000 | 0.000 |
| | 11 | 0.000 | 0.00 | 0.00 | 0.00 | 0.00 |
| | 12 | 0.000 | 0.00 | 0.00 | 0.00 | 0.00 |
| | 13 | 0.000 | 0.00 | 0.00 | 0.00 | 0.06 |
| | 14 | 0.000 | 0.00 | 0.00 | 0.00 | 0.05 |
| Mean | | 0.00 | 0.00 | 0.00 | 0.00 | 0.02 |
| SD | | 0.00 | 0.00 | 0.00 | 0.00 | 0.03 |

### Example 6

In this example, a composition containing two penetration enhancers and one solvent in combination with a wax was tested for penetration and was compared with a commercial formulation, Aldara™ 5% cream (3M Health Care Limited). The composition of the stick is presented in Table 12.

TABLE 12
Actual composition of stick used in finite dose experiment

| Batch no | ISM08164 |
|---|---|
| Ingredients | % (w/w) |
| Imiquimod | 0.09 |
| Benzyl alcohol | 34.27 |
| Propylene glycol | 14.68 |
| Isopropyl myristate | 20.97 |
| Carnauba wax | 30.00 |
| Total % (w/w) | 100.0 |

In the penetration experiment above, the recommended dose of Aldara™, 10 mg/cm² was applied for the tested compositions. Full thickness pig ear skin was used as membrane.

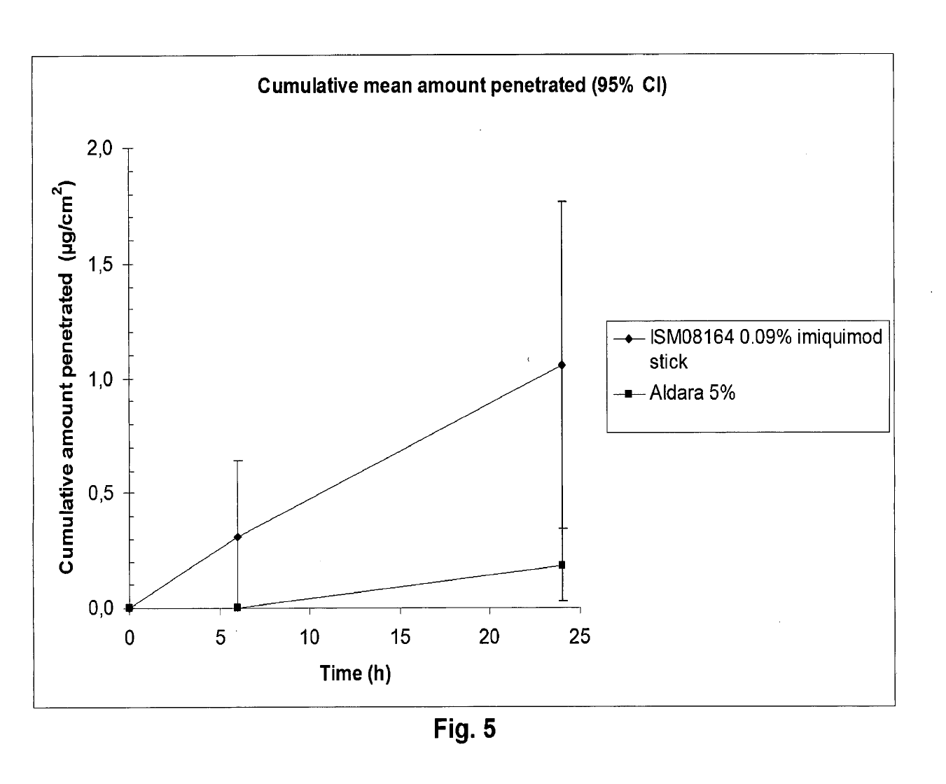

The results are shown in FIG. 5 as the mean cumulative amount imiquimod penetrated. Five cells were used for ISM08164 and four cells were used for Aldara™ 5% cream. Error bars represent 95% confidence intervals. The cumulative average amount penetrated is about 5 times higher for the invented formulation than for Aldara™ although the total dose given is 50 times lower for the novel formulation.

### Example 7

The in vitro penetration of imiquimod from Formulation H, presented in Table 13, was evaluated and compared to the penetration of imiquimod from the commercially available Aldara™ 5% cream (3M Health Care Limited).

TABLE 13
Composition of Formulation H
Formulation H

| Ingredient | Function | % (w/w) |
|---|---|---|
| Imiquimod | drug | 0.15 |
| Benzyl alcohol | S | 43.74 |
| Propylene glycol | PE | 18.80 |
| Ethyl oleate | SM | 7.11 |
| Isopropyl myristate | PE | 30.19 |

| | |
|---|---|
| Total % (w/w) | 100.00 |

The following abbreviations are used: “S” solvent, “PE” penetration enhancer, and “SM” solubility modifier.

The experimental parameters of the in vitro experiment are presented in Table 14.

TABLE 14
Experimental parameters
Example 7

| Total experimental time (h) | 48 | |
|---|---|---|
| Sampling points (h) | 6, 12, 24, 36, 48 | |
| Receptor medium | PBS buffer at pH 7.4 and Volpo | |
| | Formulation H | Aldara ™ 5% cream |
| Number of cells | 5 | 4 |
| Mean amount formulation | 63.5 | 62.6 |
| applied per cell (mg) | | |

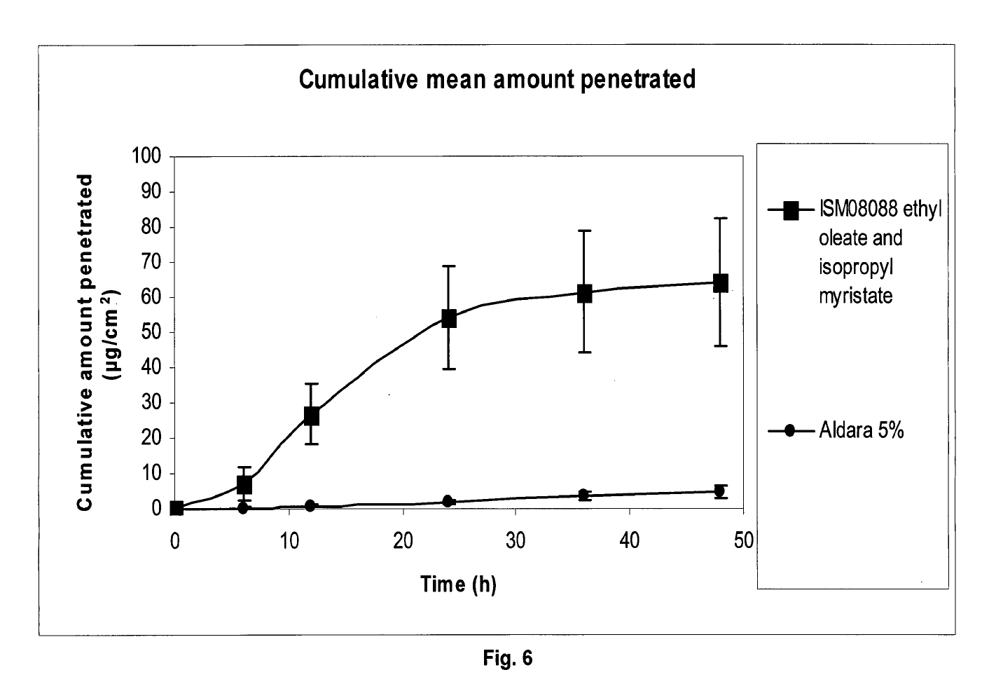

The results, presented in FIG. 6, show that the mean amount imiquimod penetrated was more than 45 times higher using Formulation H compared to the Aldara™ 5% cream. FIG. 6 shows the mean amount imiquimod penetrated per cm², n=5. Formulation H contains imiquimod, benzyl alcohol, propylene glycol, ethyl oleate and isopropyl myristate.

Comparing these results with the results presented in Example 1, it can be seen that a very high penetration increase was obtained when ethyl oleate and isopropyl myristate were added to the formulation. For Formulation H, the dose fraction imiquimod penetrated after 48 hours was approximately 40% (w/w) while for Aldara™ 5% cream it was about 0.1% (w/w).

While the claimed invention has been described in detail and with reference to specific embodiments thereof, it will be apparent to one of ordinary skill in the art that various changes and modifications can be made to the claimed invention without departing from the spirit and scope thereof.

Embodiments of the invention include, but are not limited to, the following:

1. A topical composition comprising a drug, a combination of at least two penetration enhancing agents, wherein at least one of the penetration enhancing agents is selected from the group consisting of esters of saturated or unsaturated fatty acids and lower alcohols, and iso-forms of alcohols; wherein at least one of the penetration enhancing agents is selected from the group consisting of aliphatic diols and triols; and wherein the components are present in a non-aqueous solvent system.
2. The composition according to embodiment 1, wherein the drug is a lipophilic drug.
3. The composition according to embodiment 2, wherein the drug is chosen from the group consisting of immunomodulators or immune response modifiers, tricyclic antidepressants, analgetics, anaestetics, anti-inflammatory, β blocking agents, antimicrobials and Ca blocking agents.
4. The composition according to embodiment 3, wherein the drug comprises an immunomodulating compound.
5. The composition according to embodiment 4, wherein the immunomodulating compound comprises a toll like receptor 7(TLR7) ligand.
6. The composition according to embodiment 4, wherein the immunomodulating compound is imiquimod.
7. The composition according to embodiment 1, wherein the drug is present in an amount of about 0.01 to about 5% by weight based on the total weight of the composition.
8. The composition according to embodiment 7, wherein the drug is present in an amount of about 0.01 to about 1% by weight based on the total weight of the composition.
9. The composition according to embodiment 1, wherein the alcohol is isopropyl alcohol or isobutyl alcohol.
10. The composition according to embodiment 1, wherein the penetration enhancing agents comprise isopropyl myristate and propylene glycol.
11. The composition according to embodiment 1, wherein the combined amount of penetration enhancers is from about 1 to about 99% by weight based on the total weight of the composition.
12. The composition according to embodiment 11, wherein the combined amount of penetration enhancers is from about 50 to about 99% by weight based on the total weight of the composition.
13. The composition according to embodiment 11, wherein the combined amount of penetration enhancers is from about 5 to 60% by weight based on the total weight of the composition.
14. The composition according to embodiment 1, wherein a ratio between first and second penetration enhancing agents is from about 1:10 to about 10:1.
15. The composition according to embodiment 14, wherein the ratio between first and second penetration enhancing agents is about 1:2 to about 2:1.
16. The composition according to embodiment 1, wherein the non-aqueous solvent comprises at least one selected from the group consisting of aromatic alcohols, esters of aromatic alcohols and fatty acids or esters of alpha-hydroxy acids, and short-chain alcohols having up to eight carbons.
17. The composition according to embodiment 1, wherein the non-aqueous solvent comprises at least one selected from the group consisting of cyclohexanol, diacetone alcohol, ethylene glycol monomethyl ether, diethylene glycol monomethyl ether, ethylene glycol monoethyl ether, diethylene glycol monoethyl ether, methyl salicylate, benzoic acid, oleyl alcohol, oleic acid and acetic acid.
18. The composition according to embodiment 1, wherein the solvent comprises at least one selected from the group consisting of short chain aliphatic alcohols having up to eight carbons.
19. The composition according to embodiment 18, wherein the solvent comprises at least one selected from the group consisting of ethanol, propanol and isopropanol.
20. The composition according to embodiment 1, wherein the solvent comprises at least one solvent selected from the group comprising esters of alpha-hydroxy acids and short-chain alcohols having up to eight carbons.
21. The composition according to embodiment 1, wherein the solvent or combinations of solvents are present in an amount of 1 to 80% by weight based on the total weight of the formulation.
22. The composition according to embodiment 1, further comprising a structural agent.
23. The composition according to embodiment 1, further comprising non-solvents to dilute the composition and to create texture.
24. The composition according to embodiment 1, further comprising a colouring agent.
25. The composition according to embodiment 22, wherein the structural agent comprises at least one wax selected from the group consisting of soft white paraffin and paraffin oil to increase the viscosity of the formulation.
26. The composition according to embodiment 22, wherein the structural agent comprises a soluble or non-soluble polymer.
27. The composition according to embodiment 1, further comprising a solubility modifier in amounts of about 1 to about 60% by weight based on the total weight of the composition.
28. A topical composition according to any one of the embodiments above formulated as a solution, a gel, a cream, a paste, an ointment, an oil, or a stick.

What is claimed is:

1. A topical composition comprising an immunomodulatory effective amount of imiquimod and a combination of at least two penetration enhancing agents, wherein the penetration enhancing agents comprise isopropyl myristate and propylene glycol, in a non-aqueous solvent system.
